(19) United States
(12) Patent Application Publication
(10) Pub. No.: US 2016/0008261 A1
(43) Pub. Date: Jan. 14, 2016

(54) Skin and Hair Moisturizing Composition

(71) Applicant: Mary Fields Taylor, Fort Worth, TX (US)

(72) Inventor: Mary Fields Taylor, Fort Worth, TX (US)

(21) Appl. No.: 14/794,306

(22) Filed: Jul. 8, 2015

Related U.S. Application Data
(60) Provisional application No. 62/023,231, filed on Jul. 11, 2014.

(51) Int. Cl. A61K 8/92; A61Q 17/04; A61Q 19/00; A61K 8/67; A61Q 7/00

(52) U.S. Cl. CPC A61K 8/922; A61K 8/92; A61K 8/678; A61Q 7/00; A61Q 19/007; A61Q 17/04; A61K 2800/20

(57) ABSTRACT

A skin and hair moisturizing composition. The skin and hair moisturizer composition includes coconut oil, petroleum jelly, apricot kernel oil, and vitamin E. The composition utilizes coconut oil and apricot kernel oil in order to provide an aromatic moisturizer. Additionally, the skin and hair moisturizing composition includes petroleum jelly to alleviate severely dry skin, wherein vitamin E is used to combat free-radicals resulting from exposure to the sun. The composition can be used all over an individual's body, obviating the need to buy multiple different moisturizers for different applications to the body, and helps individuals who have dry skin conditions, such as eczema. The composition can be utilized at home or can be offered for use in hair salons in order to moisturize hair.

### CROSS REFERENCE TO RELATED APPLICATION

This application claims the benefit of U.S. Provisional Application No. 62/023,231 filed on Jul. 11, 2014. The above identified patent application is herein incorporated by reference in its entirety to provide continuity of disclosure.

### BACKGROUND OF THE INVENTION

### Field of the Invention

The present invention relates to moisturizers. More specifically, the present invention provides a skin and hair moisturizing composition comprising coconut oil, petroleum jelly, apricot kernel oil, and vitamin E, wherein the composition is adapted to moisturize dry skin and strengthen hair in order to prevent breakage.

During the change of winter and summer seasons, skin and hair become dry and brittle. Dry skin is itchy and flaky, causing pain to an individual as he or she attempts to alleviate the itchiness via scratching. Furthermore, dry, flaky skin and dry, brittle hair is unattractive, which leaves an individual feeling uncomfortable and insecure. These individuals are embarrassed to leave their home or to be seen by other individuals as a result of their appearance. Therefore, individuals need moisturizing lotions or oils to help keep their skin and hair strong, moisturized, and beautiful.

Many individuals who experience dry skin attempt to utilize hydrating oils for locking in moisture over their entire body. However, these individuals, particularly individuals with dry skin conditions, such as eczema, are often required to buy multiple variations of lotions or oils in order to treat different affected areas. Therefore, there exists a need in the prior art for a moisturizer that can effectively treat both the hair and the skin of an individual.

Compositions have been disclosed in the known art that relate to moisturizing compositions. These include devices that have been patented and published in patent application publications. These compositions generally relate to a skin moisturizer or hair cream, such as U.S. Pat. No. 8,673,328 to Bloomberg, U.S. Pat. No. 3,932,611 to McCarthur, U.S. Patent Application No. 2012/0237613, U.S. Pat. No. 5,547,602 to Schuler, and U.S. Patent Application No. 2009/0130220 to Johnson. These prior art compositions have several known drawbacks. These compositions fail to provide both a skin and hair moisturizing composition comprising the combination of apricot kernel oil and vitamin E, along with a base moisturizing composition of coconut oil and petroleum jelly.

It is submitted that the present invention substantially diverges in design elements from the prior art and consequently it is clear that there is a need in the art for an improvement to existing skin and hair moisturizing compositions. In this regard the instant invention substantially fulfills these needs.

### SUMMARY OF THE INVENTION

In view of the foregoing disadvantages inherent in the known types of skin and hair moisturizing compositions now present in the prior art, the present invention provides a new skin and hair moisturizing composition wherein the same can be utilized for providing convenience for a user when protecting his or her skin and hair against dryness caused by various factors, such as changing weather.

It is therefore an object of the present invention to provide a new and improved skin and hair moisturizing composition that has all of the advantages of the prior art and none of the disadvantages.

It is another object of the present invention to provide a skin and hair moisturizing composition comprising coconut oil, petroleum jelly, apricot kernel oil, and vitamin E, wherein the composition is adapted to be topically applied to a user's skin and hair.

Another object of the present invention is to provide a skin and hair moisturizing composition wherein the composition is adapted to be stable so as to provide moisturizing effects to a user's skin and hair for an extended amount of time.

Yet another object of the present invention is to provide a skin and hair moisturizing composition that comprises one or more emollients, such as petroleum jelly and apricot kernel oil, adapted to make a user's skin softer and more pliable.

Yet another object of the present invention is to provide a skin and hair moisturizing composition further comprising a sun blocker in order to protect a user's skin and hair from the ultraviolet light of the sun.

Yet another object of the present invention is to provide a skin and hair moisturizing composition further comprising one or more perfumes in the form of essential oils in order to provide a desired fragrance to the composition.

Other objects, features and advantages of the present invention will become apparent from the following detailed description taken in conjunction with the accompanying drawings.

### DETAILED DESCRIPTION OF THE INVENTION

Reference is made herein to the attached drawings. Like reference numerals are used throughout the drawings to depict like or similar elements of the skin and hair moisturizing composition. For the purposes of presenting a brief and clear description of the present invention, the preferred embodiment will be discussed as used for moisturizing a user's skin and hair with a topical composition. The figures are intended for representative purposes only and should not be considered to be limiting in any respect.

The skin and hair moisturizing composition comprises coconut oil, petroleum jelly, apricot kernel, and vitamin E. The moisturizing composition can be provided with a number of different components, such as sun blockers and fragrances, in various quantities in order to achieve a desired result. The combination of oils and vitamins are blended together in order to provide an effective moisturizing oil. The skin and hair moisturizing composition is adapted to be applied to both skin and hair areas in order to moisturize these areas and reduce the effect of dry skin conditions, such as eczema. Furthermore, the composition is adapted to eliminate the need for user's to purchase multiple, expensive oils and lotions in order to treat both their skin and hair.

The skin and hair moisturizing composition is adapted to penetrate the top layers of skin, leaving the skin feeling soft and supple. In use, the skin and hair moisturizing composition is adapted to be applied to the body after showering. The user places a small amount of the composition in his or her hands, and rubs evenly onto his or her dry skin and hair. The composition is adapted to moisturize the skin throughout the day, thereby preventing the need to reapply thereto. The skin and hair moisturizing composition is adapted for both at-home use and professional use, such as with physical therapist offices, spas, and the like.

The skin and hair moisturizing composition comprises at least 40% coconut oil. Coconut oil comprises a high content of fat in the form of triglycerides, wherein triglycerides are used to increase the moisture content of the skin. Triglycerides are adapted to create a barrier on the surface of the skin, thereby reducing the ability of water to evaporate therefrom. Furthermore, the high fat content reduces the ability of the coconut oil to oxidize and break down. In this way, the coconut oil contained in the skin and hair moisturizing composition is stable and prevents the need to continuously reapply the composition to the skin. The triglycerides in coconut oil further reduces the protein loss in hair by coating the hair and binding to the hair proteins in order to prevent breakage thereof.

The skin and hair moisturizing composition further comprises at least 15% petroleum jelly by weight. The petroleum jelly is an emollient that prevents moisture loss of the skin. Petroleum comprises a mixture of waxes that are adapted to provide a barrier on the skin's surface, thereby sealing in the skin's moisture. Petroleum is further adapted to relieve skin itchiness and pain as a result of skin conditions, such as eczema, whereby the petroleum reduces the dryness of the skin. Furthermore, petroleum jelly is non-comedogenic and non-allergenic, thereby providing a safe means to add protection against moisture loss in the skin and hair moisturizing composition.

The skin and hair moisturizing composition further comprises at least 5% apricot kernel oil by weight. Apricot kernel oil comprises anti-inflammatory, antioxidant, and anti-aging properties when applied topically, in addition to providing an emollient to the composition. Apricot kernel oil comprises a high content of oleic and linoleic fatty acids. Oleic fatty acids are adapted to make hair and skin more pliable and softer to the touch. Linoleic fatty acids are adapted to control water loss of the hair. Apricot kernel oil is adapted to penetrate the skin and helps the skin retain elasticity, thereby preventing wrinkles to the skin. Furthermore, apricot kernel oil is readily absorbed into the skin and hair without leaving a greasy residue. Preferably, 100% organic apricot kernel oil is included in the composition. However, other embodiments comprise non-organic apricot kernel oil.

The composition further comprises at least 1% vitamin E by weight, wherein the vitamin E is adapted to provide protection against free-radicals, such as exposure to ultraviolet light from the sun. In the preferred embodiment, vitamin E is used in the form of D-ALPHA tocopheryl acetate. However, in other embodiments, any suitable form of vitamin E can be used.

In the preferred embodiment of the invention, coconut oil is present in 55 to 60% by weight, petroleum jelly is present in 20 to 25% by weight, apricot kernel oil is present in 15 to 20% by weight, and vitamin E is present in 2 to 5% by weight. In some embodiments, one or more perfumes are included in the composition, wherein each perfume comprises a fragrant essential oil. In other embodiments, the skin and hair moisturizing composition comprises a sun blocker, such as titanium dioxide, zinc oxide, and oxybenzone.

The preferred embodiment of the skin and hair moisturizing composition is prepared by measuring and weighing the desired ingredients to be used. Next, the ingredients are combined and mixed together until the desired consistency is reached. The composition is adapted to leave a user's skin and hair feeling soft and supple. Furthermore, other embodiments are adapted to be utilized as a sunscreen in addition to a moisturizer, thereby eliminating the need to purchase a variety of lotions and oils. The moisturizing composition goes on smooth and remains thereon for an extended period of time, thereby eliminating the need to keep reapplying the moisturizer to the skin and hair, thus saving the individual money.

It is therefore submitted that the instant invention has been shown and described in what is considered to be the most practical and preferred embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1. A skin and hair moisturizing composition, comprising:
coconut oil;
petroleum jelly;
apricot kernel oil; and
vitamin E.

2. The skin and hair moisturizing composition of claim 1 which comprises by weight:
40 to 70% coconut oil;
15 to 35% petroleum jelly;
5 to 25% apricot kernel oil; and
1 to 10% vitamin E.

3. The skin and hair moisturizing composition of claim 1 which comprises by weight:
55 to 60% coconut oil;
20 to 25% petroleum jelly;
15 to 20% apricot kernel oil; and
2 to 5% vitamin E.

4. The skin and hair moisturizing composition of claim 1, further comprising one or more sun blockers.

5. The skin and hair moisturizing composition of claim 1, further comprising one or more perfumes.

6. The skin and hair moisturizing composition of claim 1, wherein vitamin E is in the form of D-ALPHA tocopheryl acetate.
